(19) United States
(12) Patent Application Publication
(10) Pub. No.: US 2011/0295151 A1
(43) Pub. Date: Dec. 1, 2011

(54) Enteroendocrine Manipulation for Metabolic Effect

(76) Inventors: Gregory J. Bakos, Mason, OH (US); Edward G. Chekan, Cincinnati, OH (US); Denzel Z. Herrera-Davis, Cincinnati, OH (US); Jason L. Harris, Mason, OH (US); Gary L. Long, Cincinnati, OH (US); Rudolph H. Nobis, Mason, OH (US); Mark S. Ortiz, Milford, OH (US); Mark D. Overmyer, Cincinnati, OH (US); David N. Plescia, Cincinnati, OH (US); Randy J. Seeley, Cincinnati, OH (US); William B. Weisenburgh, II, Maineville, OH (US); Tamara S. Vetro Widenhouse, Clarksville, OH (US); Mark S. Zeiner, Mason, OH (US); Andrew M. Zwolinski, Hamburg (DE)

(21) Appl. No.: 13/104,282

(22) Filed: May 10, 2011

Related U.S. Application Data
(60) Provisional application No. 61/348,265, filed on May 26, 2010.

(51) Int. Cl. A61M 37/00; A61P 3/00; A61K 9/00; A61F 2/04; A61K 49/00; A61P 3/04; A61B 10/02; A61M 5/00; A61K 35/55; A61K 9/20

(52) U.S. Cl. 600/567; 424/93.7; 424/400; 424/439; 424/464; 424/9.5; 424/9.1; 604/22; 604/522; 600/37; 604/8

(57) ABSTRACT

L-cells may be introduced in the gastrointestinal tract. L-cells are used in the digestive process to produce a more efficient and lasting means of regulating feelings of satiation in a patient. Desired metabolic effects may be achieved by manipulating L-cells via delivery sites, frequency of delivery, or type of biological substance delivered.

### PRIORITY

This application claims priority to U.S. Provisional Patent Application Ser. No. 61/348,265, entitled “Enteroendocrine Manipulation for Metabolic Effect,” filed May 26, 2010, the disclosure of which is incorporated by reference herein.

### BACKGROUND OF THE INVENTION

This invention relates to systems and methods for controlling feelings of satiation. In particular, this invention relates to systems and methods for regulating enteroendocrine cells such as L-cells in optimizing gastrointestinal processing of food.

Several hormones are responsible for regulating body weight. These hormones are released from regions of the gastrointestinal tract or neuromuscular system in response to stimulation by the presence of absence or food in the body. In some patients, genetic or anatomic anomalies can result in excess weight gain. In many cases, it is the over- or under-stimulation of appetite-controlling hormones that result in excess weight gain.

Methods of regulating these hormones by pharmaceutical means have been proposed. However, many of these pharmaceutical approaches have proven less than adequate in meeting the needs of overweight or obese patients. Additionally, the intestinal brake concept has been used to resect a segment of ileum and reposition it at a location earlier in the gastrointestinal tract. The process has the effect of stimulating the L-cells earlier and speeding the effects of satiation. There is a need for more effective methods and devices for regulation of hormones responsible for body weight without drastic anatomical changes.

### BRIEF DESCRIPTION OF THE DRAWINGS

FIG. 1 is a perspective view of a stent.

FIG. 2 is a perspective view of an implanted stent.

FIG. 3 is side view of an implanted stent in the intestines.

FIG. 4 is a perspective view of an anchor.

FIG. 5 is a side view of the mucosa of the bowel with implanted anchors.

FIG. 6 is perspective view of an implant in a portion of a bowel.

FIG. 7 is side view of an implant in the gastrointestinal tract.

FIG. 8 is side view of an implant in the gastrointestinal tract.

FIG. 9 is side view of an implant in the gastrointestinal tract.

FIG. 10 is side view of an implant in the gastrointestinal tract with a gastric coil.

FIG. 11 is a perspective view of a porous implant for L-cell delivery.

FIG. 12 is a cross-sectional view of the porous implant for L-cell delivery.

### DETAILED DESCRIPTION

The following detailed description is intended to be representative only and not limiting as to devices and methods for manipulating L-cells to produce a desired change in the metabolic status of a patient. Those of ordinary skill in the art will understand that the devices and methods specifically described herein and illustrated in the accompanying drawings are non-limiting, exemplary embodiments and that the scope of the present invention is defined solely by the claims. The features illustrated or described in connection with one exemplary embodiment may be combined with the features of other embodiments. Such modifications and variations are intended to be included within the scope of the invention as presented herein.

Modification to Existing Cells

In one method for manipulating L-cells, L-cells along the gastrointestinal tract of a patient are modified to achieve improved metabolic effect. In a first example, early initiation of intestinal brake is achieved by modification of L-cells in the gastrointestinal tract to alter metabolism. In a second example, L-cells are mutated to modify their reproduction so that the cells proliferate unnaturally. The cells are then spread to additional parts of the gastrointestinal tract and affect satiation at desired time intervals. In a third example, the intestinal brake procedure is initiated. However, instead of complete resection of the ileum to move the L-cells, the physician performs an endoscopic submucosal dissection (ESD) procedure to remove large portions of the mucosal lining that contains the L-cells from the ileum during a colonoscopy after intubating the ileum. That tissue is subsequently implanted back into the patient at a location downstream of the duodenum, such as via an enteroscopy procedure. For this relocation procedure, the physician may intubate the duodenum with a longer enteroscope or a single/double-balloon endoscope. The physician also performs another ESD at this location to remove the existing mucosa. In an alternative means to traverse the entire small bowel, an over-tube endoscopy system, such as a the Endo-Ease® Endoluminal Advancement System available from Spirus Medical of Stoughton, Mass., is used. This over-tube type of system enables physicians to perform flexible endoscopy procedures more efficiently.

To perform the ESD, an explant area is inflated by a submucosal needle before removal by a snare device. Ultrasonic endoscopy may be used to insure that dissection is completed to an accurate tissue depth. Once dissection is completed, the explanted mucosa from the ileum is fastened in places by means of sewing, clipping, or gluing as examples of fastening means and not by way of limitation. In this manner, the mucosa with the L-cells is then located in a more advantageous position to promote satiation. The explant may also be used to patch the ileum. The procedure may be optimized via a series of experiments to determine the best position and/or length of replanted segment that is most effective. This procedure is less invasive than performing an intestinal transposition procedure.

Implantation of Cells

In an additional embodiment of the present invention, cells are moved without removing a segment of bowel or mucosal lining. A biopsy is performed of L-cells from the ileum. The L-cells are then cultured in a dish for a period of time. The cultured L-cells are then implanted into the same patient during a subsequent procedure at a more desired location to promote satiation. The procedure is minimally invasive and does not require breaching of the intestinal wall.

The implantation of the cultured L-cells is performed in a number of exemplary procedures. In a first example, the desired area is abraded with a brush to initiate a healing response. The abraded L-cells are then placed or injected to the desired area. In a second example, a patch containing L-cells is used to cover the abraded area much like a bandage. The patch may be L-cell-infused or an absorbable mesh used as a scaffold for L-cell growth. An example of this is disclosed in US2005/0131386A1, Jun. 16, 2005 “METHOD AND DEVICE FOR MINIMALLY INVASIVE IMPLANTATION OF BIOMATERIAL”, which is hereby incorporated herein by reference.

Specialized devices for the biopsy of L-cells from the gastrointestinal tract allow access to L-cells using minimally invasive or non-invasive means. Embodiments of L-cell biopsy techniques and devices are presented herein. In a first embodiment, areas of concentrated L-cells are located within a patient before biopsy is performed. Once located, a map of the L-cells is created, and the biopsy procedure is performed with guidance from the map. In a second embodiment, modified nutrients are first provided to the patient. The nutrients are modified with markers that appear in imaging equipment. As the modified nutrients are absorbed by the L-cells, imaging of the patient occurs and the locations of the L-cells are pinpointed. In an exemplary marking technique, fluorescent marking is used to tag the L-cells so that the cells are easily activated under direct visualization. This provides the surgeon more immediate feedback as to the location and boundaries of the target area. In a third embodiment, an ultrasonic endoscope is used to locate areas of high L-cell concentration. L-cells are open-type cells and therefore may have different reflective properties. The wavelength of the imaging signal is specially tuned to recognize the difference between closed and open cells. Then, the imaging system is used as a guide to harvest the cells at the appropriate tissue depth.

Enhancements to the aforementioned embodiments may also be incorporated. In a first enhancement example, the patient consumes designated types of food for a period of time that hyper-stimulate L-cell production. This permits the surgeon to take biopsies at times that L-cell concentration is relatively high or at a maximum. In a second enhancement example, an endoscope is outfitted with a linear display and a biopsy needle. The endoscope is used to insert a biopsy needle into the lumen to a visualized depth. This system and method is used to visualize biopsy layers of L-cells ensuring that the physician inserts the biopsy needle to the correct depth.

Techniques in L-cell culturing insure that healthy, high yield L-cells are implanted into the patient. Alternatively, implantation may not be required. As an example, a patient may take a pill laced with L-cells to introduce enough cells to make the desired response. L-cells may be harvested from animals such as pigs or other animals. L-cells harvested from animals are treated to regulate the immune response of the patient. This provides a less costly alternative than harvesting and culturing L-cells from a human. Additionally, animal L-cell harvesting provides more L-cells than a human donor.

Culture of Tissue from Healthy Individuals.

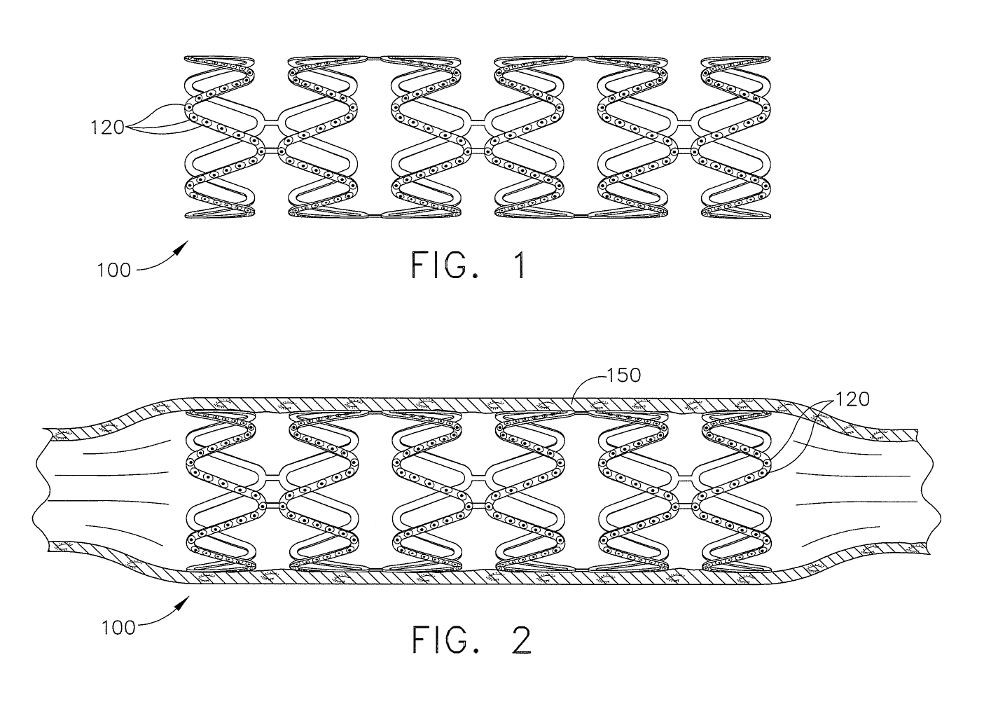

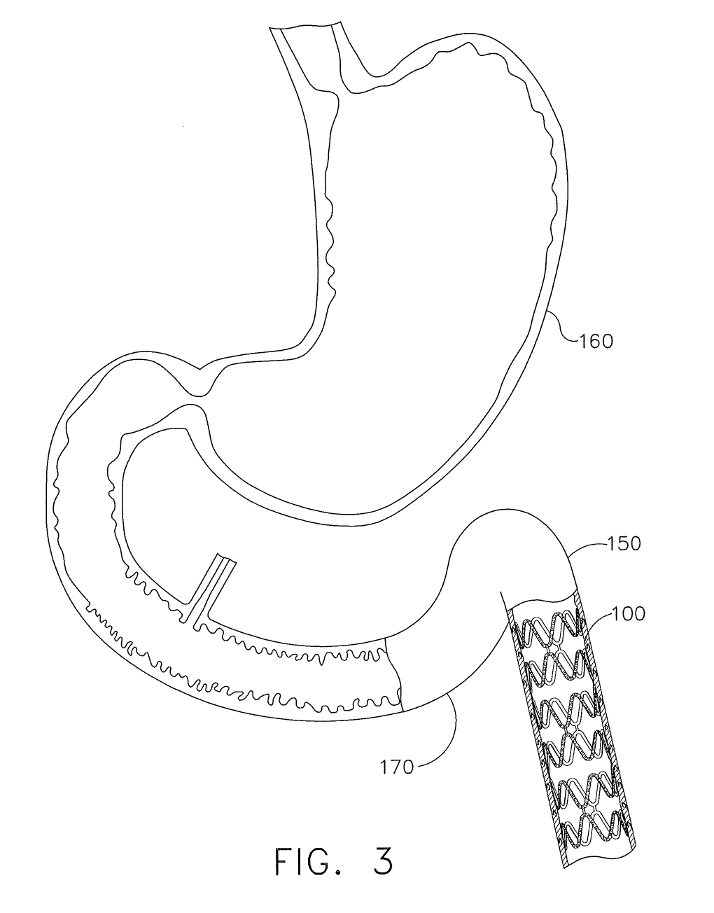

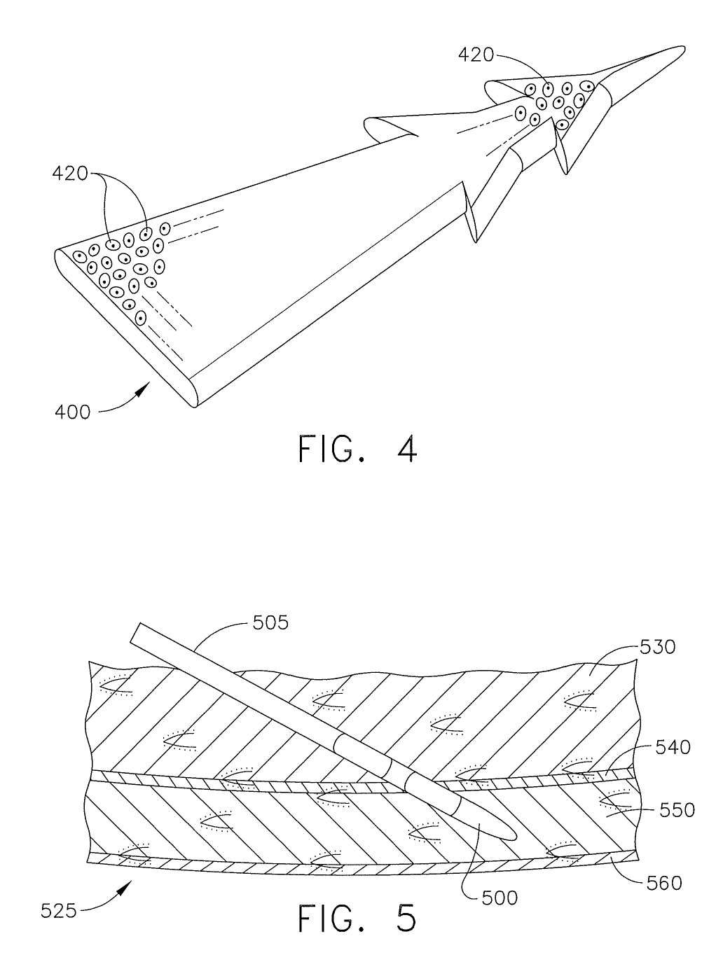

Turning now to FIGS. 1-3, in another series of embodiments of the present invention, devices, systems, and methods are disclosed to extend the active residence time or increase the frequency of secretory pulses of GLP-1 or any other native or synthetic chemical or hormone active within and upon the gastrointestinal tract able to be secreted by a mammalian cell (e.g. cholecystokinin (CCK), oxyntomodulin, PYY, insulin, glucagon, vasoactive intestinal polypeptide (VIP), insulin-like growth factors (IGF's), etc.). Collection and culture of desired cell types (e.g. intestinal L-cells, I-cells, or S-cells), their progenitor cells, or other mammalian cells transformed or transfected to produce the desired peptide may be used to regulate residence time or increase secretory pulsing. For example, the cells may be grown in vitro on an endoscopically deployable, biodegradable device such as an expandable polymer stent 100 having a similar geometry as the intended site of deployment. The stent 100 may be composed of a biocompatible material such as poly-L-lactic acid (PLLA), polyglycolic acid (PGA), poly(D, L-lactide/glycolide) copolymer (PDLA), polycaprolactone (PCL) collagen or alginate.

Similarly, cells 120 of the gastrointestinal tract may be harvested and cultured in vitro or well as seeded and grown in culture on three-dimensional scaffold material. The seeded device 100 is endoscopically placed within the lumen of a target organ (e.g. stomach 160, duodenum, or small intestine 170) and the cells 120 remain protected during deployment either mechanically and/or chemically. The cultured cells 120 remain attached, functional, and continue secreting desired peptides intraluminally and in a paracrine manner initially. As the scaffold device is epithelialized and incorporated into walls 150 of the segment or lumen, they secrete in an endocrine manner additionally.

The cells remain sensitive to the normal signals that precipitate peptide synthesis and release cells (fat, glucose, fatty acids, etc.). Alternatively, cells derived from stem cell cultures may be a source for transplantation. In addition, cells may be obtained for the use in scaffold culturing from tissue donors. Donors are generally selected based on healthier metabolic ability or profiles.

The stent device 100 offers several advantages. The device 100 may be placed completely endoscopically in a non-invasive manner. The size of the device 100 and biofunctional capacity of the cells 120 may be tailored to surgical preferences and the patient's physiological needs. The device 100 may be used in the treatment of a number of diseases such as diabetes, obesity, and pancreatic insufficiency by selection of a cell type for seeding onto the stent and/or the genes the cells are transformed with prior to seeding. Further, the device 100 is biodegradable and resorbable permitting short-term regulation of therapy at intervals determined by the treating physician. In addition, the device may be used stand-alone or adjunctively with current bariatric procedures such as gastric banding, gastric bypass, and sleeve gastrectomy. Once implanted, the device provides a nutrient-responsive, biological source of one or more active gastrointestinal peptides, thus shortening the time to satiation, prolonging satiety, and increasing insulin sensitivity depending on the peptide product(s) selected.

Intestinal Anchors with L-Cells

Turning now to FIGS. 4, 5A, and 5B, in another embodiment, tissue anchors 400, 500 are used as a culture scaffold for L-cells 420. A device 505, preferably a multifire, is used to implant the anchors 400, 500 in the mucosa 530 of the bowel 525. The anchors 400, 500 dissolve, leaving L-cells that are incorporated into the surrounding tissue including the mucosa 530, submucosa 540, and muscle layers 550. The serosa 560 is not penetrated to prevent complications associated with bowel penetration. The surface of the anchor 400, 500 is coated with L-cells 420. The anchor 400, 500 may be imbedded in the tissue deep enough to expose some or all of the cells to existing vasculature. In this manner, the cells may be permanently translated and the transfer of hormones occurs directly to the blood stream.

A scaffold and a device for placing such a scaffold, such as the device described in patent publication U.S. 2005/0131386 to Freeman et al. may be used to culture L-cells and to place the L-cell culture into the optimal location within the mucosa of the bowel. Such a scaffold may be implanted proximal to the ileum so as to position L-cells in an area stimulated by upstream food passage. Alternatively, a soft-tissue implant such as described in patent publication U.S. 2005/0142161 to Freeman et al. may be implanted in an area proximal to the ileum.

Fly Paper Substrate

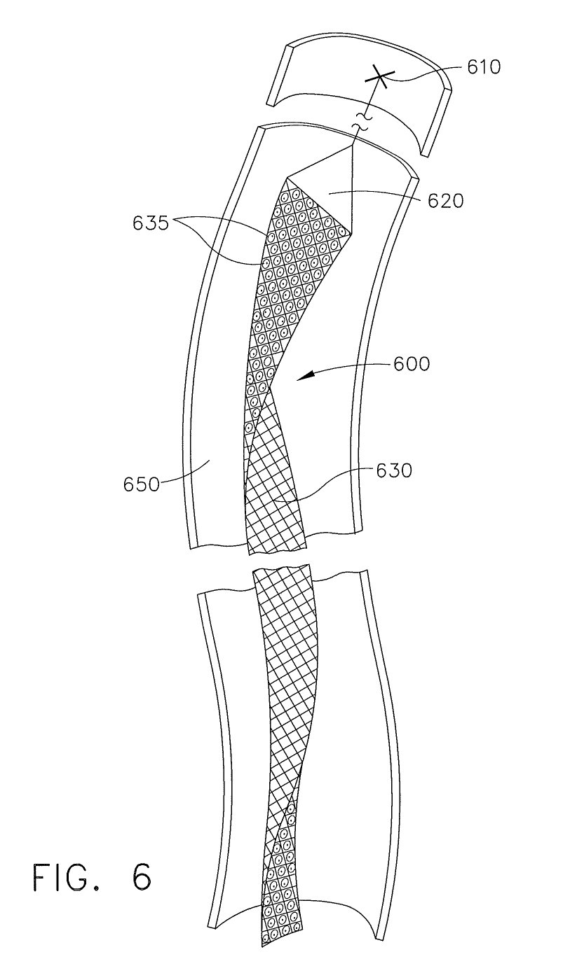

Turning now to FIGS. 6-10, other embodiments of an implantation method include storage of the cells on an initially coiled strip. The strip is coiled into a tube and positioned into a desired implant location. Once the tube portion is anchored within the bowel, the coiled strip is pulled from the tube, exposing the L-cells contained on the strip. In an exemplary embodiment, the uncoiling of the strip occurs without action of a health professional and is driven by natural peristalsis, as an example. The distal end of the substrate is attached to a larger object such as a ball and wound or compressed so that as peristaltic waves push the ball through the digestive tract, the substrate is deployed in the desired manner. In effect, the substrate doesn't remain in the stomach. The ball may be digestible or otherwise comprised of a material that reduces or otherwise breaks into passable size. Alternatively, the ball may be initially sized for natural passage from the body. In still another example, fasteners such as tacks, staples, sutures, T-tags, and similar fastening devices are used to affix the object to one end in the stomach and permit it to unroll into a desired shape.

In an example as seen in FIG. 6, the implant 600 is comprised of three sections: an attachment section 610, an extension section 620, and a substrate section 630. The attachment section 610 maintains the position of the implant 600 within the bowel lumen 650. The extension section 620 acts to distance the cells from the attachment section 610. The length of the extension 620 is adjustable to fit the anatomy of the patient pre-operatively or post-operatively by using an endoscope. The substrate section 630 contains the cells 635 as well as any substances necessary to prolong the active life of the cells 635. Examples of such substances include nutrients and pharmaceuticals.

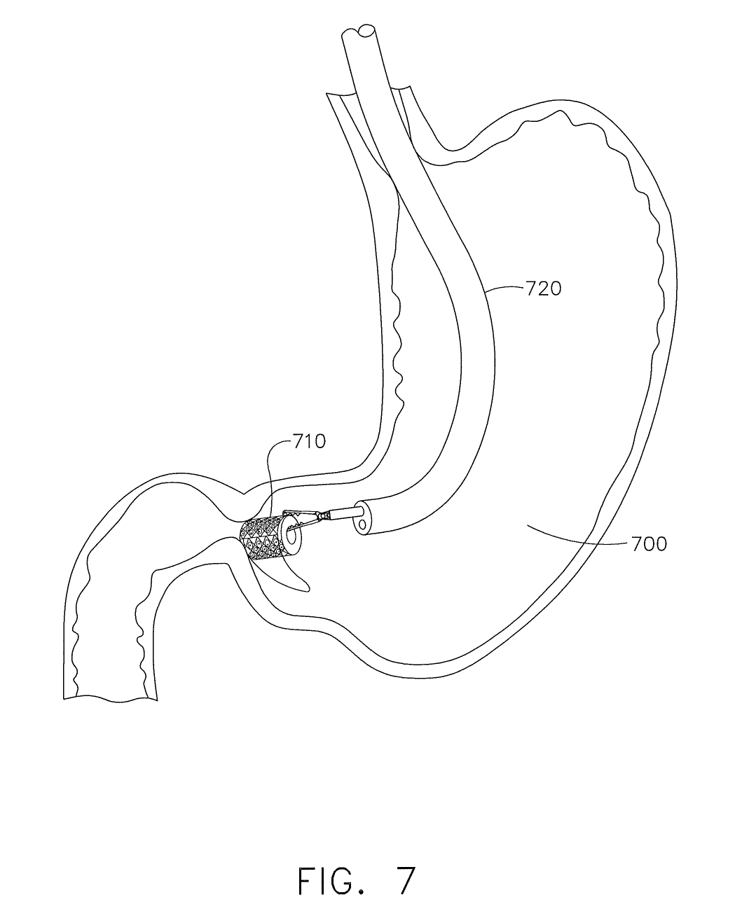

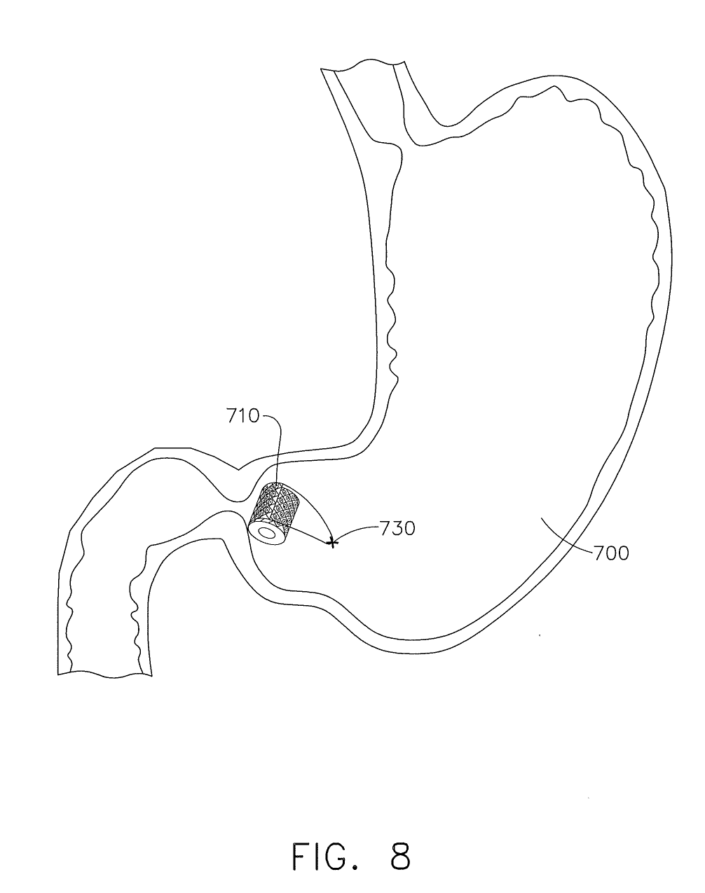

In the example of FIGS. 7-9, the implant 710 is a coiled substrate introduced into a region of the stomach 700 through a natural orifice such as the mouth or rectum. Introduction and positioning of the implant/substrate 710 may be accomplished by an endoscope 720. A proximal end of the substrate 710 is fixtured within the gastrointestinal tract at an attachment point 730 within the stomach 700. Suture t-tags, staples, or other tissue-fastening means may be used. Also, the fixturing means may be designed with biodegradable materials to dissolve away or otherwise release when the active life of the implanted cells is over. The implant 710 is then allowed to pass.

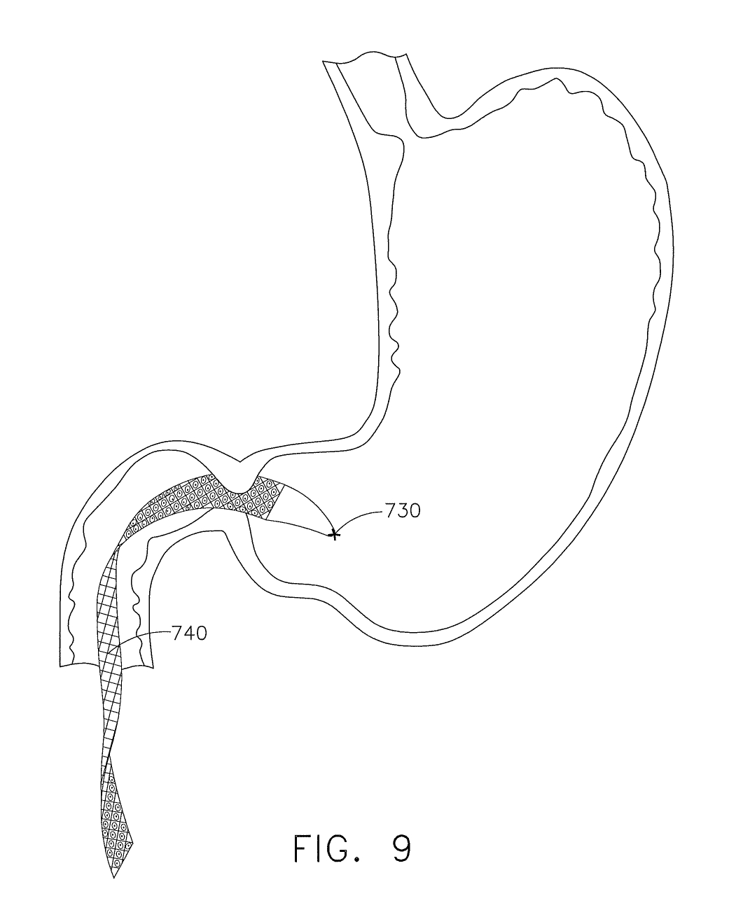

Once fixtured, substrate 740 is allowed to uncoil as shown in FIG. 9. The distal end of the substrate 740 may be attached to a large object such as a ball and wound or compressed so that as peristaltic waves seek to push the ball through the digestive tract, the substrate 740 is deployed in the desired manner (e.g., it doesn't remain in the stomach). The ball may be biodegradable, digestible, or otherwise comprised of a material that reduces or breaks into passable size. Alternatively, the ball may be initially sized for natural passage from the body.

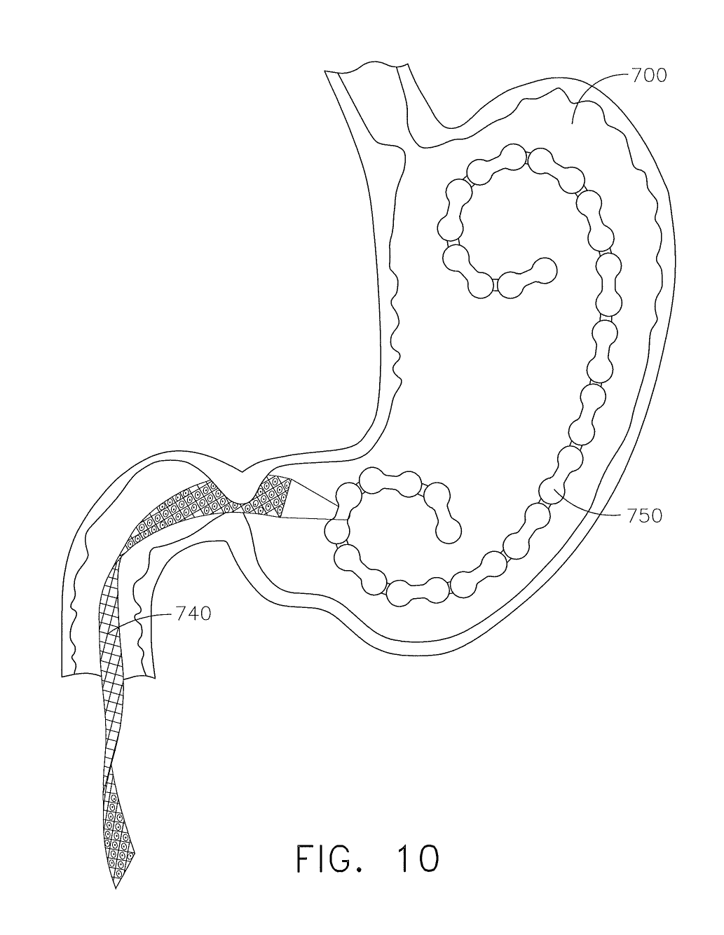

In an alternative embodiment shown in FIG. 10, a gastric coil 750 as described in published U.S. Pat. Application No. 2008/0058840 to Albrecht et al., incorporated herein by reference in its entirety, is used as a mounting platform for an implanted substrate 740. During implantation, the substrate 740 is attached to the gastric coil 750. Attachment methods include the use of sutures, staples, and latches on the substrate 740, coil 750, or both. The distal end of the substrate 740 may be attached to a large object such as a ball and wound or compressed such that as peristaltic waves push the ball through the digestive tract, the substrate 740 is deployed in a desirable manner and does not remain in the stomach. The ball may be digestible or otherwise comprised of a material that reduces or breaks into passable size. Alternatively, the ball is initially sized for natural passage from the body.

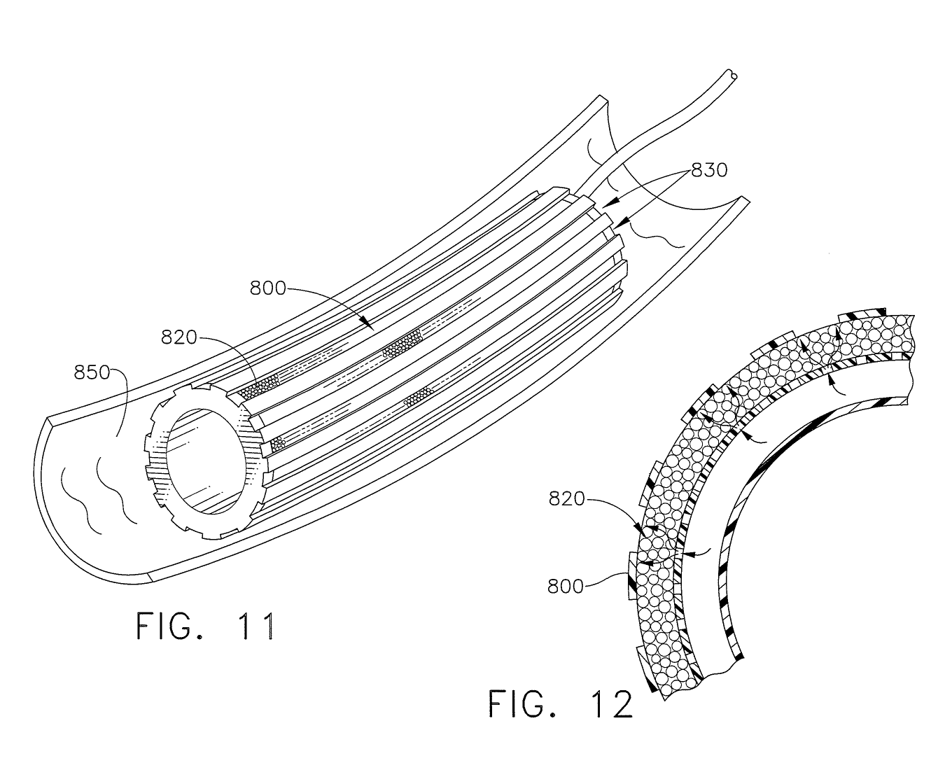

Additional embodiments shown in FIGS. 11 and 12 disclose implants with means to replenish the supply of L-cells, support substance for L-cells, or both. A porous material provides a substrate 800 for L-cells 820 in contact bowel 850 contents. Internal channels 830 allow fluid introduced through a catheter 850 to be distributed to the porous surface. In one example, support substances are provided to L-cells to prolong the life of the cell colony. These substances may include nutrients, pharmaceuticals, and the like. In another example, new L-cells are introduced by removal or rejection of old L-cells. In still another example, a mixture of L-cells and support substances are provided. The catheter 850 may extend through the lumen directly, or through the biliary tree and further through the liver. Other embodiments may include a nasogastric tube in place of a subcutaneous fill port.

In some settings, such as in the case of non-absorbable implants, the scaffolding used as a growth and transplant media may cause complications by virtue of being an implant. Thus, it may be desirable to have a growth media that is implantable and absorbable, allowing the cultured cells to grow in to the new environment. In some versions, such a growth media may comprise keratin. With high sulphur content and a blend of amino acids including cysteine, phenylalanine, leucine, glutamine and lysine, keratin proteins perform a fundamental structural role in nature. Fractions of keratin proteins and internal lipids may be extracted from pure New Zealand wool using a gentle process that maintains protein and lipid integrity. By reconstituting these biopolymer materials, distinct and highly functional characteristics of the original proteins and lipids can be captured and used in different product types and applications. Keratin is normally non-soluble and cannot be digested or absorbed by the human body. However, it is possible to extract keratin fractions in a soluble and digestible form, leaving the natural amino acid structure intact and therefore potent. Thus, keratin extracts may be used as a building block. The material can then be implantable and absorbable by the body. Using such material in an implantable medical device may provide an enzymatic type of absorption, such that water has no effect on the life of the product. In other words, a keratin based device may be unaffected by humidity, which might otherwise result in problems with shelf life. In addition, the material may be gamma sterilized, unlike some other biopolymers such as PGA and PLA which cross link and lose strength. Furthermore, the material may be injection molded for a variety of structural components. The tissue healing response to keratin may take place throughout the structure of the implant, as opposed to at the tissue/implant interface. Keratin may also accelerate tissue healing. As a natural structural element, keratin is capable of cooperating with the body's healing mechanisms. It may be noted that, as the thickness of the keratin material is a factor in degradation rate, the rates of degradation of elements of the system may be programmed by controlling thickness of the keratin at key points, such as desired detachment points, such that passage of a large clump of balloons simultaneously may be prevented.

Transplant of L-Cells from a Healthy Individual

In a further series of embodiments of the present invention, gastrointestinal tissue from a “healthy” weight person is resected. The resected tissue is placed into an obese patient. The healthy resected tissue contains the cells responsible for secreting regulated levels of hormones which in turn lower the BMI of the obese recipient. Transplanted tissue may include portions of the gastrointestinal tract including duodenum, ileum, gastric tissue, or esophageal tissue. The unhealthy tissue of the obese patient does not require resection if it is demonstrated that the healthy transplanted tissue maintains greater control over hormones.

In one embodiment, an entire bowel segment is transplanted to the patient. In another embodiment, a biopsy of the cells is taken from the donor, grown on an implant, and implanted into the patient. The cells may also come from multiple healthy people. A mixture of the cells could be created that most suits the patient. L-cells derived from stem cell cultures may also be a source for transplanting. Using these embodiments, hormones may be manufactured normally by the body without the need for invasive pharmaceuticals.

Variations on the disclosed embodiments include the introduction of flora to the intestinal tract that responds to nutrients with the release of the same or similar hormones to those of L-cells. In an example, flora is engineered to respond sensitively to specific substances that affect the gastrointestinal tract. A special culture of flora may be created for an individual patient. The flora used in the culture is obtained from multiple sites such as individuals from different geographic locations, cultural backgrounds, or similarly from animals of different breeds and geographic locations. In addition, flora found in mammals that aid in the production of L-cells may be transplanted into patients. Further, custom grown flora from donor L-cells may be used. The donor may be the actual patient or could be a donor.

In still other embodiments, a patient eats food laced with L-cells. The food is custom created with harvested L-cells from a donor. L-cells may also be cultured in certain foods such as yogurt and sauerkraut.

One skilled in the art will appreciate further features and advantages of the invention based on the above-described embodiments without deviating from the scope and spirit of invention as disclosed. Accordingly, the invention is not to be limited by what has been particularly shown and described, except as indicated by the appended claims. All publications and references cited herein are expressly incorporated herein by reference in their entirety.

I/We claim:

1. A method of causing a change in a metabolic status of a patient, the method comprising:
a. harvesting L-cells from said patient at a first location within a gastrointestinal tract of the patient;
b. culturing said L-cells such that a cell culture is created; and
c. implanting said cell culture into said patient at a second location within the gastrointestinal tract of the patient to cause a change in the metabolic status of said patient, wherein the second location is upstream of the first location.

2. The method of claim 1 wherein said first location is the ileum and said second location is downstream of the duodenum.

3. The method of claim 1 comprising the step of abrading said second location prior to said implanting step, wherein said abrading step initiates a healing response.

4. The method of claim 1 wherein said L-cells are mutated to achieve an effect selected from at least one of improving metabolism, increasing ability to proliferate and spread to additional parts of said gastrointestinal tract after said implanting step, or combination thereof.

5. The method of claim 1 wherein said L-cells are cultured on a substrate and wherein said substrate containing said L-cells is implanted into said patient.

6. The method of claim 5 wherein said substrate comprises a keratin or collagen matrix

7. The method of claim 1 wherein said L-cells are placed in a patch or a scaffold prior to said implanting step.

8. The method of claim 1 wherein said L-cells are placed in a bioabsorbable patch prior to said implanting step.

9. The method of claim 1 wherein said L-cells are placed on a delivery vehicle selected from a food, a pill, a biodegradable substrate, an endoscopically deployable substrate, a polymer stent, and combinations thereof.

10. The method of claim 1 wherein said L-cells are placed on a delivery vehicle comprising a biocompatible material selected from at least one of poly-L-lactic acid, polyglycolic acid, poly(D,L-lactide/glycolide) copolymer, polycaprolcactone, collagen, alginate, or combination thereof.

11. The method of claim 1 comprising the step of locating areas of high L-cell concentration using an ultrasonic endoscope.

12. The method of claim 1 comprising the step of administering to said patient a modified nutrient, wherein said modified nutrient is capable of being absorbed by said L-cells to permit imaging of a location of said L-cells.

13. The method of claim 1, wherein said patient consumes a food type known to hyperstimulate L-cell production prior to said harvesting step.

14. The method of claim 1 wherein said harvesting step occurs during a colonoscopy.

15. The method of claim 1 wherein said harvesting step comprises using an endoscope outfitted with a linear display and a biopsy needle.

16. The method of claim 1 wherein said method is used in conjunction with a bariatric procedure selected from gastric banding, gastric bypass, sleeve gastrectomy, and combinations thereof.

17. A method of causing a change in a metabolic status of a patient, the method comprising:
a. harvesting L-cells from a mammal;
b. culturing said L-cells such that a cell culture is created;
c. placing said cell culture onto an implant, wherein said implant comprises a tube having a substrate comprising L-cells of said cell culture coiled therein; and
d. introducing said implant into a gastrointestinal tract of said patient;
wherein said implant is positioned into a bowel of said patient;
wherein said substrate comprising L-cells is exposed by the effects of natural peristalsis on said implant.

18. The method of claim 16 wherein said implant further comprises an attachment section, an extension section, and a substrate section.

19. The method of claim 16 wherein said L-cells are cultured from an individual having a healthy weight.

20. A method of extending the active residence time or increasing the frequency of secretory pulses of a native or synthetic chemical or hormone active within or upon the gastrointestinal tract of a patient, the method comprising:
a. collecting a cell type for a patient based on the metabolic disease of said patient, wherein said cell type secretes a chemical or hormone selected from cholecystokinin, oxyntomodulin, PYY, insulin, glucagon, vasoactive intestinal polypeptide, insulin-like grown factors, or combinations thereof;
b. culturing said cell type on an endoscopically deployable and degradable device; and
c. implanting said endoscopically deployable and degradable device comprising said cell type in said patient.
